(19) United States
(12) Patent Application Publication
(10) Pub. No.: US 2015/0025348 A1
(43) Pub. Date: Jan. 22, 2015

(54) Assessing Coagulation

(71) Applicants: Eric GRABOWSKI, Wellesley, MA (US); THE GENERAL HOSPITAL CORPORATION, Boston, MA (US)

(72) Inventor: Eric Grabowski, Wellesley, MA (US)

(21) Appl. No.: 14/239,055

(22) PCT Filed: Aug. 22, 2012
(86) PCT No.: PCT/US12/51866

§ 371 Date: Sep. 25, 2014

Related U.S. Application Data
(60) Provisional application No. 61/526,043, filed on Aug. 22, 2011.

(51) Int. Cl. G01N 33/49; A61B 5/154; A61B 5/15

(52) U.S. Cl. CPC G01N 33/4905; A61B 5/15003; A61B 5/154; A61B 5/150992
USPC 600/369

(57) ABSTRACT

Devices and methods for assessing coagulation include a channel configured such that shear stress, in a first portion of the channel, applied to blood drawn through the channel by a vacuum-tube attached to an outlet of the channel approximates physiological shear stresses.

### TECHNICAL FIELD

This invention relates to assessing coagulation.

### BACKGROUND

Platelet function testing can be performed using Born-O'Brien aggregometry. Born-O'Brien aggregometry remains a labor-intensive exercise, requiring advance scheduling in the coagulation laboratory, hours of technician time, and blood volumes of 15-20 ml. This instrument, moreover, utilizes re-calcified citrated platelet rich plasma (no red cells or white cells), and does not take into account the high shear rates prevalent in arteries and arterioles, vessels most likely to lead to significant blood loss following trauma.

### SUMMARY

The devices and methods described below can be used to rapidly measure bleeding and clotting risk in trauma victims without an external power source. Bedside devices to rapidly measure bleeding and clotting risk in trauma victims can be powered by a standard vacuum-containing blood tube for real-time use with small blood-volumes. The devices use a standard vacuum-containing blood tube to draw blood platelets via a needle stick through a specially designed microfluidic test chamber. The time at which blood flow is stopped by clotting in the devices provides a measure of bleeding risk. Subsequently, a fluorescence microscope can provide an assessment of platelet deposition from a glass cover slip exposed to flowing blood in the devices, and also the presence on these platelets of clot-inducing microparticles rich in the clotting activity known as “tissue factor” (TF). The approach includes the use of controlled microfluidics, simple venipuncture with a downstream Vacutainer® as “power source,” and platelet adhesion/aggregation on a collagen-coated glass cover slip (a part of the device) to quantify (using “off line” immunofluorescence) both platelet deposition in flowing blood and co-localization with platelets of circulating TF-positive microparticles.

In some aspects, devices for assessing coagulation includes a channel configured such that shear stress, in a first portion of the channel, applied to blood drawn through the channel by a vacuum-tube attached to an outlet of the channel approximates physiological shear stress. Embodiments can include one or more of the following features.

In some embodiments, the channel includes: a flow chamber; and a resistance path downstream of the flow chamber, the resistance path configured such that the shear stress in the flow chamber applied to blood drawn through the channel by the vacuum-tube is between about 1 and 100 dynes per square centimeter (e.g., above 5, above 10, above 15, below 75, below 50, and/or below 25 dynes per square centimeter). In some cases, the flow chamber has a flow chamber width between about 1 to 8 mm (e.g., 4 mm, above 2 mm, above 3 mm, above 4 mm, below 7 mm, below 6 mm, and/or below 5 mm) and a flow chamber height between about 200 and 350 microns (e.g., about 290 microns, above 225 microns, above 250 microns, above 275 microns, below 325 microns, and/or below 300 microns). In some cases, the resistance path has a resistance path width between about 0.1 and 2 mm (e.g., about 0.45 mm, above 0.25 mm, above 1 mm, below 1.5 mm, below 1 mm, and/or below 0.75 mm) and a resistance path height between about 0.1 and 2 mm (e.g., about 0.45 mm, above 0.25 mm, above 1 mm, below 1.5 mm, below 1 mm, and/or below 0.75 mm). The resistance path can have a length between about 5 and 15 centimeters (cm) (e.g., about 10 cm, above 7 cm, above 8, cm, above 9 cm, below 14 cm, below 13, cm, below 12 cm, and/or below 11 cm).

In some embodiments, the channel is configured such that, for healthy subjects, occlusion of the channel by platelet thrombus occurs after less than 0.5 to 5 ml of blood are drawn into the device.

In some embodiments, the channel is configured such that, for subjects in shock, occlusion of the channel by platelet thrombus does not occur before more than 5 ml of blood are drawn into the device.

In some embodiments, the shear stress, in the first portion of the channel, applied to blood drawn through the channel by a vacuum-tube is between about 1 and 100 dynes per square centimeter (e.g., above 5, above 10, above 15, below 75, below 50, and/or below 25 dynes per square centimeter).

In some embodiments, the shear rate, in a first portion of the channel, applied to blood drawn through the channel by a vacuum-tube are between about 100 and 10,000 per second (e.g., above 200, above 250, above 300, above 400, below 5,000, below 2,500, below 1,000, below 750, and/or below 700 per second).

In some embodiments, the channel is configured such that shear stress, in the first portion of the channel, applied to blood drawn through the channel by a vacuum tube whose volume is less than or equal to 10 ml (e.g., a 3.0 ml, a 4.5 ml, or 10 ml vacuum-tube) attached to the outlet of the channel approximates physiological shear stress.

In some embodiments, the channel is defined at least in part by a polymer layer bonded to a glass slide.

In some aspects, methods include: drawing blood into a channel by attaching a vacuum tube to an outlet of the channel; observing a length of time that passes until flow of blood through the channel is occluded; and characterizing a condition of a patient from whom the blood was drawn based at least in part on the length of time that passes until blood flow through the channel is occluded.

Embodiments can include one or more of the following features.

In some embodiments, methods also include observing degrees of platelet adhesion and aggregation in the channel until the flow of blood through the channel is occluded.

In some embodiments, methods also include detecting and quantifying tissue factor positive microparticles on platelets attached to surfaces of the channel.

In some embodiments, methods also include comparing the length of time that passes until blood flow through the channel is occluded with a total volume of the vacuum tube.

In some embodiments, methods also include drawing blood is limited to drawing less than 5 ml of blood into the device.

In some aspects, kits include devices for assessing coagulation as described above and a vacuum source. Embodiments can include one or more of the following features.

In some embodiments, the vacuum source is a vacuum tube.

In some embodiments, kits also include a butterfly needle.

In some embodiments, kits also include an angiocatheter.

A “vacuum tube” is a standard vacuum-containing blood tube rather than tubing attached to external vacuum source.

In some instances, the devices can be used to predict which critically ill patients are in greatest need of critical care resources, whether in the hospital or on the battlefield. For example, the devices and methods can be used to rapidly predict: 1) bleeding risk in hemorrhagic shock, owing to the platelet dysfunction which accompanies acidemia, hypoxia, and blood-loss anemia; 2) need for transfusion therapy in such patients (whole blood vs. packed red blood cells, platelets, plasma); and 3) likelihood of disseminated intravascular coagulation (DIC) and/or occlusive deep venous thrombosis (DVT) due to a prothrombotic state which paradoxically can co-exist with hemorrhagic shock. For example, a prothrombotic state can exist due to tissue factor (TF) in brain tissue suddenly exposed to the circulation, or to TF generated by infection and sepsis.

The devices and methods described herein can provide a rapid bedside approach to the measurement of platelet function that can be performed on-demand (rather than pre-scheduled). It is anticipated that the clinical team will be able to perform these assessments bedside because these devices and methods are less labor-intensive than, for example, conventional Born-O'Brien platelet aggregometry. Moreover, these devices and methods take into account the high blood flow velocity gradients (“shear rates”) prevalent in arteries and arterioles, vessels most likely to lead to significant blood loss following trauma. Assessments performed using these devices and methods can be performed using small volumes of blood (e.g., 0.5 to 5 ml) and, thus, can be used for patients in shock as well as newborns and infants.

These devices and methods can provide a rapid, simple, low blood volume device or method which incorporates flowing whole blood. In contrast to devices that utilize whole blood drawn through a fine capillary tube to form platelet aggregates that occlude an opening in a membrane coated with collagen and epinephrine, or collagen and adenosine diphosphate (ADP), the devices and methods described herein are sensitive to platelet hyperfunction and are intended for the bedside.

These devices and methods can also provide a measurement of the extent to which circulating tissue factor (TF) microparticles (MPs) can fuse with the platelet membrane of surface-adherent platelets. The circulating TF can co-localize with growing mural platelet thrombi (Balasubramanian et al., Blood, 100:2787-92, 2002). Circulating TF has recently become recognized not only as the initiator of primary hemostasis, but also, in pathologic states (including trauma and deep venous thrombosis), as the driving force for thrombosis and disseminated intravascular coagulation (DIC). In the circulation system, TF exists in the form of TF-positive MPs, defined as structures of diameter less than 1 to 1.5 μm that express phosphatidylserine, as determined by annexin V-binding, and TF. Levels of circulating TF-positive MPs are increased in a number of disease states, including sepsis, deep venous thrombosis (DVT), atherosclerosis, diabetes, sickle cell disease, and acute coronary syndromes (e.g., Kushak et al., 2005). Thus, devices and methods described herein can provide a measurement of platelet function that is anticipated to be able to assess both increased or decreased platelet function per se as well as the level of circulating TF. In contrast, Born-O'Brien aggregometry and the PFA-100 are insensitive to states of platelet hyperaggregability.

These devices and methods do not require use of an anticoagulant thus leaving levels of ionized calcium largely intact. These devices and methods avoid geometric irregularities in the flow path, in contrast to the PFA_{—}100, which utilizes puncture of the membrane in a cartridge of ADP or epinephrine. These devices and methods incorporate actual hematocrits, blood pH, and temperature, thereby accounting for effects of shock on these parameters. These devices and methods allow direct imaging of platelet adhesion/aggregation and provide a truly point-of-care approach.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a schematic illustration of a device for assessing coagulation.

FIGS. 1B and 1C are cross-sections of portions of a channel defined by the device of FIG. 1A.

Like reference symbols in the various drawings indicate like elements.

### DETAILED DESCRIPTION

Coagulation Assessment Device

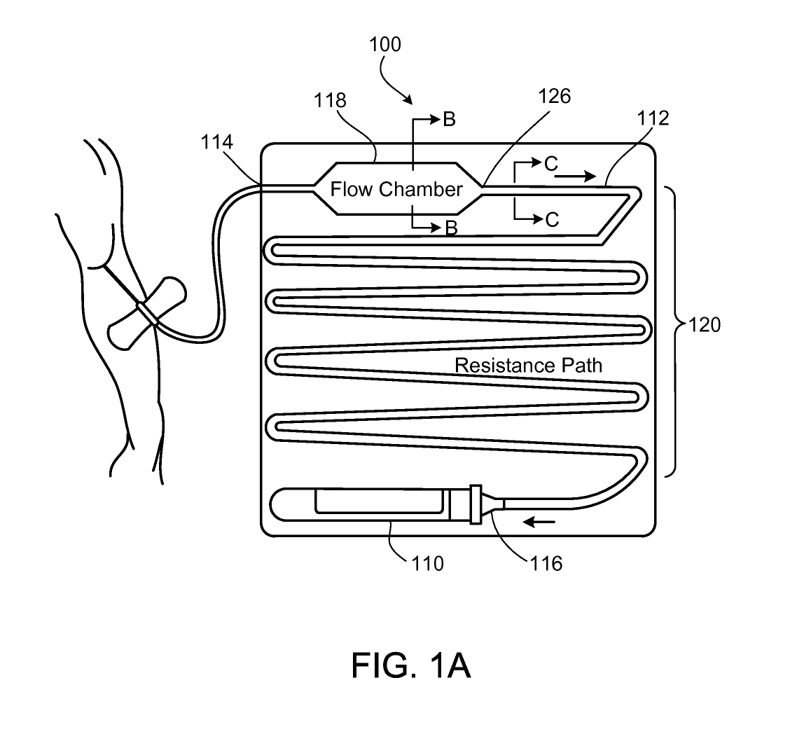

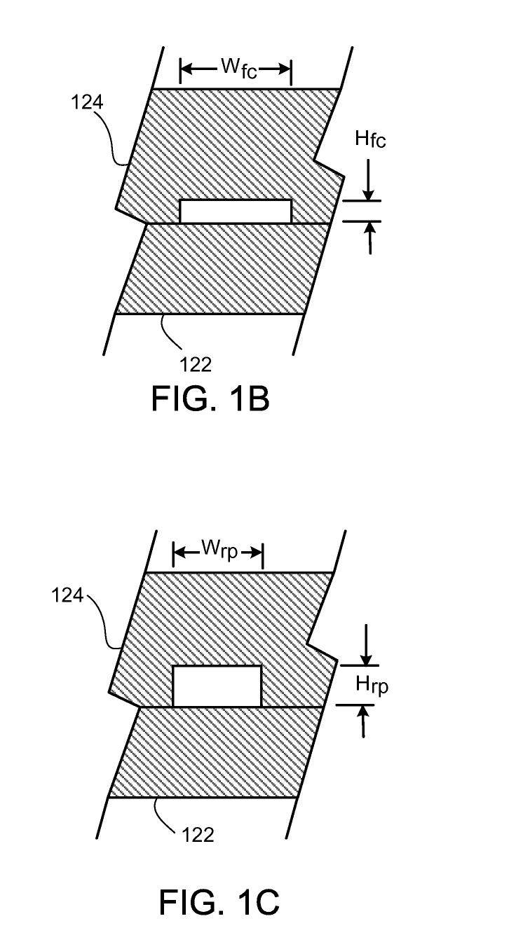

A hand-held microfluidics device 100 as shown in FIGS. 1A to 1C draws blood from a patient or normal test subject by simple venipuncture and constitutes a flowing, whole blood platelet aggregometer. The device 100 simulates blood flow in an arteriole, with controlled, arteriolar-like shear rates, and measures the ability of blood to form platelet-fibrin thrombi under controlled flow conditions. The endpoint of the simulation is when a localized part of the flow path becomes filled with thrombus occluding the blood flow.

The device 100 is powered by a standard vacuum-containing blood tube 110 such as, for example, a VACUTAINER® vacuum tube commercially available from Becton Dickenson Diagnostics, Franklin Lakes, N.J. USA 07417. The device 100 defines a channel 112 extending from a channel inlet 114 to a channel outlet 116. The channel 112 includes a flow chamber 118 and a resistance path 120. The channel 112 is defined between a substrate 122 and an overlying media 124. The substrate 122 and the overlying media 124 are preferably both clear such that they transmit light during the imaging processes described below. In the exemplary device, the substrate 122 is a glass slide and the overlying media 124 is a poly-dimethyl siloxane (PDMS) part bonded to the glass slide. A smooth transition is created from the cylindrical geometry of the venipuncture needle and upstream tubing to the parallel-plate geometry adjacent to the glass cover slip. Microfibrillar collagen is applied to substrate 122. In some embodiments, the substrate can also include, for example, tissue-factor expressing endothelium, subendothelium, certain biomaterials on glass or even plastic slides in place of the microfibrillar collagen-coated glass slide. In some embodiments, the overlying media is formed of a hard plastic (e.g., zeonor) which exhibits less hydrophobicity than PDMS.

The resistance path 120 is sized such that the shear stress/rates applied to blood drawn through the channel 112 by the vacuum-tube 110 approximate physiological shear stresses and shear rates in the flow chamber 118. The shear stresses and rates are a function of fluid and channel characteristics.

The flow chamber 118 has a rectangular cross-section having a height H_{fc}, a width W_{fc}, and a length L_{fc} extending from the flow chamber outlet 126 to the channel outlet 116. The flow chamber height H_{fc}, width W_{fc}, and length L_{fc} are chosen to provide shear stresses and shear rates that approximate physiological shear stresses (10-25 dynes/cm²) and shear rates (300-700 sec⁻¹). The sensitivity of device 100 to platelet function can depend upon a judicious choice of flow path thickness which is anticipated to be on the order of 200-300 μm. Experiments can be carried out with various flow path thicknesses, from 250 μm to 400 μm, and with decreasing widths, in order to optimize sensitivity of the occlusion time to platelet deposition on the glass cover slip. This occlusion time reflects the use in the present device of non-anticoagulated blood and a collagen-coated glass cover slip with controlled flow conditions everywhere adjacent to the cover slip. In contrast, the “closure time” of the PFA-100 is produced using citrated blood and a hole punched in a collagen-coated filter impregnated with ADP or epinephrine, with controlled flow conditions upstream.

The cross-sectional shape and length of the resistance path limit the velocity and flow rate of fluid flowing through the system. The device 100 can have a resistance path 120 with a rectangular cross-section having a height Hᵣₚ, a width Wᵣₚ, and a length Lᵣₚ extending from the flow chamber outlet 126 to the channel outlet 116. To reduce the total size of the device 100, the resistance path can be arranged in a serpentine pattern with gradual transitions. In some embodiments, the resistance path 120 can have other cross-sectional shapes (e.g., be formed from circular tubing). The height Hᵣₚ, width Wᵣₚ, and length Lᵣₚ of the resistance path 120 are chosen to provide overall fluid flow rates through the device 100 such that a fluid velocity of approximately 150 millimeters/second (mm/s) to approximately 500 mm/s (e.g., more than 25 mm/s, more than 50 mm/s, more than 75 mm/s, more than 100 mm/s more than 200 mm/s, less than 3000 mm/s, less than 2500 mm/s, less than 2000 mm/s, less than 1500 mm/s, less than 1000 mm/s, less than 750 mm/s, less than 600 mm/s, less than 400 mm/s, and/or less than 300 mm/s) though the flow chamber 118. The overall resistance can be decreased (and flow rate increased) by increasing the cross-sectional area and/or decreasing the length of the resistance path.

Using a KCl solution to simulate blood density, VACUTAINER vacuum tubes have been observed to have a “fill time” of the order of 4 to 12 seconds, depending upon VACUTAINER vacuum tube size (3.0, 4.5, and 10 ml). The fill time for a 3.0 ml tube is 4.52±0.41 sec (mean±SD, N=5), while for a 4.5 ml tube it is 5.21±0.19 sec, and for a 10 ml tube it is 11.78±2.81 sec. These values for a 4.5 ml tube are lower than the 7-8 sec observed for a phlebotomist withdrawing blood from a normal subject into three 4.5 ml tubes. The difference is likely due to the greater viscosity of whole blood (normally 3 to 5 centipoise) as compared to that (about 1.0 centipoise) for the KCl solutions. The vacuum pressure force is such that the starting pressure estimated in the tubes at sea level at 23° C. is 0.2 atmospheres, based upon the fraction of a given tube which eventually fills with blood. This degree of vacuum is confirmed by Dr. David Warunek, World Wide Director for Scientific Affairs for Becton Dickenson Diagnostics who provided an estimate of 0.22 atmospheres. Nonetheless, the flow rate achieved with a 10 ml Vacutainer is about 50-60 ml/min for a KCl solution for 12 seconds, and, correcting for viscosity, about 12-15 ml/min for 48 seconds for whole blood.

Mathematically, the flow rate, F, through the device 100 is given by

F=−dV/dt=kV0e^{-kt},

where V is the remaining fill volume, k is a constant with units of sec⁻¹, V0 is the fill volume, and t is time. Therefore, the volume of blood, V ml, drawn into the Vacutainer becomes

V=V0(1−e^{-kt}),

These relationships assume that blood flow is Eulerian (inertia dominant), Newtonian, that the upstream tubing (e.g., between the patient and the device) does not contribute to the effective tube volume during the tube filling time, and that the upstream venous pressure does not drop to a level leading to vein closure, the “critical closing pressure” determined by the ability of a vein to withstand a net pressure tending to force its collapse. For a fill time, using a KCl solution, of 12 sec, k is 0.25 sec⁻¹ in order for kt to be of the order of 3 half-lives, for which F at t=0 is 2.5 ml/sec. For whole blood with a viscosity 3-5 times that of crystalloid (cell-free buffered salt solution), k is approximately 0.06 sec⁻¹, F at t=0 is 1 ml/sec, or 60 ml/min, and the fill time is about 48 sec, in agreement with the observations discussed above.

However, a lower flow rate of about 2 ml/min for 1-3 minutes is needed for the present microfluidics device to achieve physiological shear stresses (10-25 dynes/cm²) and shear rates (300-700 sec⁻¹) as well as levels of platelet adhesion/aggregation which are reproducible and informative. Calculations which assume a fully-developed Poiseuille flow of blood in a channel of rectangular cross-section suggest that a channel approximately 10 cm in length and 450 μm×450 μm in cross-section, with a short section (e.g., flow chamber 118) measuring 250 μm×1250 μm in rectangular cross-section (to accommodate the glass slide; see below) will produce the desired flow rate. The corresponding Reynolds number for this flow is on the order of 9.2, so that there is no danger of generating turbulence. Furthermore, with a shear stress of approximately 25-30 dynes/cm², there is no concern for hemolysis as the blood flows through the system. In order to minimize the total volume of the device, the 10 cm length of channel can be comprised of five 2-cm lengths, arranged in a serpentine pattern with gradual transitions (channel radius of curvature approximately five channel heights). Thus, the entire channel can easily fit in the 2.2 cm×6.0 cm area of a standard glass cover slip.

The purpose of the flow chamber 118 with a 250 μm×1250 μm cross-section (aspect ratio of 10:1) is two-fold. First, such a section places the greatest localized resistance to flow (and greatest surface shear rates) adjacent to the collagen-coated cover slip, which promotes the formation of occlusive platelet thrombus at this site. Calculations show that the short section will have a relative pressure gradient (pressure drop per unit length of channel) of 1.95 compared to that for the square channel. Second, a parallel-plate geometry will make for ease of calculation of surface shear rates and shear stresses from standard formulae.

Methods of Manufacture

The channel 112 can be manufactured from poly-dimethyl siloxane (PDMS) using soft lithography with patterned wafers (e.g., SU-8, MicroChem, Boston, Mass.) with the PDMS part being bonded to a glass slide as described by Chung et al. (2009). Using soft lithography with SU-8 (MicroChem) patterned wafers to transfer the characteristics of the microfluidic channel from a computerized data file allows for precise control of flow path dimensions as well as economy of production of large numbers of identical chambers. When combined with cell culture protocols, microfluidic devices can be formed which use small quantities of reagents and cells, have specified local shear rates and shear stresses, and permit high resolution imaging of cellular events in real time (Vickerman et al, 2008).

Several approaches can be used to attach the glass cover slip to the rest of the microfluidics device. One approach is to bond the PDMS to a glass slip (Chung et al, 2009). Another is to “seal” the glass slip by means of a hand vacuum pump to bring the glass into firm apposition to the soft PDMS, as has been done previously using a soft Silastic gasket (Grabowski, 1990).

The same geometry fashioned from PDMS can be produced in Zeonor or other standard hard plastic using the hot embossing method. In hot embossing, a negative master is made from the original silicon wafer used for PDMS fabrication. Initially, the master can be produced from one of the PDMS imprints, using epoxy. For later mass production, the master can be made of metal for greater durability and stability.

A special construction consideration is the creation of a smooth transition from a cylindrical geometry (that of the venipuncture needle and upstream tubing) to a parallel-plate geometry adjacent to the glass cover slip. Streamlining can help avoid unwanted platelet thrombus formation at the inlet and exit headers, especially since the blood will be non-anticoagulated. The transition in the exemplary device 100 is between a needle with circular cross-section and inner diameter 600 μm, to the 10 cm-long channel measuring 450 μm×450 μm in cross-section. Inlet and outlet cross-sectional areas can be designed to equal that of the connecting needle, with a gradual 3-dimensional transition, using a molded plastic element, over a distance of 1 cm to the cross-sectional area of the resistance channel. Complex micro-geometries can be formed with injection molding, even including the use of “powders” of ceramics or stainless steel particles (4 μm) together with binder systems (Liu et al, 2003). The channel depth can be decreased from 600 μm to 250 microns and its width increased from 600 μm to 1250 μm in a second smooth transition at the flow chamber 118, again using a molded 3-dimensional plastic element, to a localized 1 cm-long section adjacent to the glass cover slip. Computational fluid dynamics can be used to confirm that the transition is free of any trapped vortices or regions of unusually low or high shear stress.

Operation

Bedside devices to rapidly measure bleeding and clotting risk in trauma victims can be powered by a standard vacuum-containing blood tube for real-time use with small blood-volumes. The devices use a standard vacuum-containing blood tube to draw blood platelets via a needle stick through coagulation assessment device 100. The time at which blood flow is stopped by clotting in the devices provides a measure of bleeding risk. Subsequently, a fluorescence microscope can provide an assessment of platelet deposition from a glass cover slip exposed to flowing blood in the devices, and also the presence on these platelets of clot-inducing microparticles rich in the clotting activity known as “tissue factor.”

Clotting or Occlusion Time Endpoint

The primary endpoint is flow device occlusion by platelet thrombus, with cessation of blood flow into the Vacutainer. An occlusion time of 6-9 min is expected to represent the approximate range with normal blood. Blood with hypofunctioning platelets or even diminished platelet number, as in DIC, is expected to have an occlusion time of 9-12 min, or even 12 min or greater, depending upon the degree of hypofunction.

Longer occlusion times (e.g., >9 min) can indicate diminished platelet function, and vice-versa. A secondary endpoint is degree of platelet adhesion/aggregation on the slide up to the occlusion time. Platelet adhesion/aggregation is intrinsically a dynamic process which is shear-rate and time-dependent (Grabowski, 1972; Grabowski, 1990). For more refined information regarding platelet function, the glass slide can be removed and platelet deposition on this slide, together with co-localized TF-positive microparticles, assessed “off line” using two-color fluorescence microscopy in concert with simple digital image analysis.

Another secondary endpoint is the detection and quantification of TF-positive

MPs on platelets on the glass cover slip. The importance of such circulating TF is believed to lie in the augmentation and propagation of platelet adhesion/aggregation, and its existence has been reported for laser-induced injuries in the microcirculation of the mouse cremaster muscle (Falati et al, 2003; Chou et al, 2004). Using real-time imaging, we have shown that circulating TF can co-localize with growing mural platelet thrombi (Balasubramanian et al., 2002). As a consequence, the measurement of platelet function by our approach should be able to assess both increased or decreased platelet function per se as well as the level of circulating TF. In contrast, Born-O'Brien aggregometry and the PFA-100 are insensitive to states of platelet hyperaggregability. Our flow system, via platelet-fibrin thrombus formation, therefore has the potential to be sensitive to increases in circulating TF, as in patients in a prothrombotic state following massive trauma.

The cover slip is removable from the flow device by relieving the vacuum holding in place the glass slide and removing the glass slide while the device is immersed in saline (to neutralize forces of surface tension, which otherwise can keep the slide firmly adherent). Platelets on the glass slide can be labeled by addition of Alexa 555-conjugated CD41 Fab fragments or Alexa-555-conjugated TAB; TF with Alexa 488-conjugated rabbit anti-human TF antibody. Both the Alexa 350-conjugated anti-human fibrin antibody above and an Alexa 488-conjugated anti-fibrinogen antibody (F4639, Sigma-Aldrich, St. Louis, Mo.) can be used to distinguish deposition of fibrin, recognized by both antibodies, from binding/adsorption of fibrinogen, recognized by only the second antibody. Platelet adhesion/aggregation on the collagen surface can be imaged using 400× epifluorescence videomicroscopy, with quantitation of adherent platelet aggregates via digital images obtained using, for example, a CoolSnap HQ camera with 12-bit (4095 gray level) resolution. The images can be processed, for example, using SimplePCI core software. Platelet aggregates can be counted in 20 continuous view fields (each field 435 μm×580 μm). An argon-krypton three-line laser can provide the fluorescent light for excitation at the appropriate wavelengths, with a filter wheel mounted on the excitation source to provide for the delivery of monochromatic light. This can 1) quantify platelet adhesion/aggregation in terms of percent pixels occupied by platelets, and quantify platelet volume, and 2) observe and quantify TF co-localization via two-color fluorescence, with cumulative TF fluorescence activity being normalized by that for adherent platelets. Platelet volume can be calculated using the integrated fluorescence intensity over the pixels occupied by all the platelet aggregates in a specified field of view, with a calibration based upon the integrated fluorescence intensity for a monolayer of platelets obtained using a concentration of citrate anticoagulation which blocks platelet aggregation but not platelet adhesion.

The dyes chosen are all from the Alexa family (Molecular Probes, a subsidiary of Invitrogen). These dyes are generally more stable, more intense, and less pH-sensitive than most standard dyes having similar excitation and emission wavelengths. The specific antibodies chosen for Alexa dye conjugation include for platelets the TAB monoclonal mouse anti-human antibody (McEver et al, 1980) and anti-CD41 Fab fragments; for TF protein, rabbit polyclonal and mouse monoclonal anti-human TF antibodies; and for fibrin, NYB-T2G1, Accurate Scientific, Westbury, N.Y. The feasibility of labeling platelets and not affecting their function in whole blood has been shown previously (Grabowski, 1990).

A simplified version of this fluorescence imaging apparatus can be implemented retaining only the salient features of the above. Such a simplified apparatus can be made available for the follow-up glass cover slip studies via use of a more centralized hospital or battlefield location. For example, the glass slide can be removed from the chamber, washed and fixed in paraformaldehyde, incubated with fluorescently-labelled antibodies directed against platelet GPIIb, tissue factor, and/or fibrinogen, and then examined using a fluorescence microscope.

Device endpoints can be correlated with the results of parallel, more routine coagulation assays (e.g., the ACT), as well as with clinical scoring indices (e.g., MODS, ISS, PIM2, PRISM; see later) of trauma severity and/or critical illness and, consequently, likely bleeding risk and transfusion need. We will validate the instrument by testing it first in normal subjects, and subsequently in a series of trauma patients. Comparison of outcome measurements (time to occlusion of blood flow, platelet deposition, and TFaccumulation) will be made with an outcomes score which measures multi-organ dysfunction and trauma severity, the activated clotting time (ACT), the PT and PTT, and, in select cases, thromboelastography and Born-O'Brien platelet aggregometry.

This can correlate findings with a score documenting the overall impact on mortality of multi-organ dysfunction, trauma to body regions, and/or clinical and laboratory parameters (MODS and APACHEII, with supplementation by the ISS, for adult patients, and PIM and PRISM for pediatric patients), thereby providing clinical confirmation of the utility of the measurements of platelet function and platelet-associated tissue factor. These scores are described below. While these scores do not specifically address bleeding risk and transfusion need, they surprisingly actually do predict risk of bleeding and transfusion, as shown for the ISS by Como et al (2004) and Eastridge et al (2006). Bleeding risk undoubtedly correlates with trauma severity.

MODS (the Multiple Organ Dysfunction Score—Marschall et al, 1995) is a score predictive of hospital and ICU mortality, and is the sum total of a score of zero to 4 for six organ systems: respiratory, renal, hepatic, cardiovascular, hematologic, and neurologic. The score therefore ranges from zero to 24, with a monotonic increase in mortality with increasing score. A score of 13-16, for example, is associated with a 50% mortality.

APACHE II (the Acute Physiology and Chronic Health Evaluation II—Knaus et al, 1985) was designed to measure the severity of disease and predict mortality for patients greater than 15 years admitted to ICUs. A point score (zero to 71) is calculated from 12 physiologic measurements, including blood pressure, body temperature, and heart rate, together with information about previous health status and, more recently, the principal diagnosis leading to ICU admission.

ISS (the Injury Severity Score—Baker et al, 1974) is an anatomical scoring system that provides an overall score for patients with multiple injuries. Each injury is assigned an Abbreviated Injury Scale score which is identified with one of six body regions: head, face, chest, abdomen, extremities (including pelvis), and external. Only the highest MS score in each body region is used. The three most severely injured body regions then have their score squared and added together to produce the ISS score, values for which can range from zero to 75. The score correlates linearly with mortality, morbidity, and hospital stay. The MGH SICU automatically calculates the ISS for each patient in its care, and these scores will be made available to the applicant.

PIM2 (the Pediatric Index of Mortality 2—Slater et al, 2003) is a score predicting mortality for children in intensive care. It is based upon admission circumstances (elective or not), underlying condition, response of pupils to bright light (>3 mm and both fixed), need for mechanical ventilation (at any time during first hour in the ICU), excess of systolic blood pressure over 120 mmHg, base excess (mmHg in arterial or capillary blood), and FiO2 (%)/PaO2 (mmHg). The score is calculated using a logit formula and specified weightings of each of the above parameters.

PRISM (the Pediatric Risk of Mortality score—Pollack et al, 1988) is also calculated using a logit formula. However, it is based upon a greater number of clinical and laboratory parameters: systolic blood pressure, diastolic blood pressure, heart rate, respiratory rate, PaO2/FiO2, PaCO2, PT and PTT, total bilirubin, serum calcium, serum potassium, plasma glucose, and plasma HCO3-, pupillary reactions, and Glasgow (neurological) score.

Using this microfluidic device with a VACUTAINER vacuum tube as power source can provide a simulation of physiologic blood flow with an easily observable primary endpoint: occlusion of blood flow at a time in minutes calibrated to the VACUTAINER vacuum tube blood volume (zero to 10 ml) achieved before occlusion.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### LIST OF REFERENCES

1. S. P. Baker et al. The Injury Severity Score: A method for describing patients with multiple injuries and evaluating emergency care. J. Trauma 14:187-196, 1974.

2. V. Balasubramanian, E. F. Grabowski, A. Bini, and Y. Nemerson. Platelets, circulating tissue factor, and fibrin colocalize in ex vivo thrombi: Real-time fluorescence images of thrombus formation and propagation under defined flow conditions. Blood 100:2787-92, 2002.

3. J. Chou, N. Mackman, G. Merrill-Skoloff, B. Pedersen, B. C. Furie, and B. Furie. Hematopoietic cell-derived microparticle tissue factor contributes to fibrin formation during thrombus propagation. Blood 104:190-97, 2004.

4. S. Chung, R. Sudo, P. J. Mack, C. Wan, V. Vickerman, and R. D. Kamm. Cell migration into scaffolds under co-culture conditions in a microfluidic platform. Lab Chip 9:269-275, 2009.

5. I. J. Como et al. Blood transfusion rates in the care of acute trauma. Transfusion 44:809-813, 2004.

6. I. Del Conde, C. N. Shrimpton, P. Thiagarajan, J. A. Lopez. Tissue-factor-bearing microvesicles arise from lipid rafts and fuse with activated platelets to initiate coagulation. Blood 106:1604-11, 2005.

7. B. J. Eastridge et al. Early predictors of transfusion and mortality after injury: A review of the data-based literature. J. Trauma 60:S20-S25 (suppl 6), 2006.

8. S. Falati, Q. Liu, P. Gross, G. Merrill-Skoloff, J. Chou, E. Vandendries, A. Celi, K. Croce, B. C. Furie, and B. Furie. Accumulation of tissue factor into developing thrombi in vivo is dependent upon microparticle P-selectin glycoprotein ligand 1 and platelet P-selectin. J Exp Med 197:1585-98, 2003.

9. E. Fressinaud, K. S. Sakariassen, C. Rothschild, H. R. Baumgartner, and D. Meyer, Shear rate-dependent impairment of thrombus growth on collagen in nonanticoagulated blood from patients with von Willebrand disease and hemophilia A. Blood 80:988-994, 1992.

10. P. L. Giesen, U. Rauch, and B. Bohrmann. Blood borne tissue factor: another view of thrombosis. Proc. Natl Acad Sci 96:2311-5, 1999.

11. E F Grabowski. Momentum and Mass Transport in Flowing Blood with Application to Thrombus Formation, doctoral dissertation, Columbia University, February 1972.

12. E. F. Grabowski. Platelet aggregation in flowing blood at a site of injury to an endothelial cell monolayer: Quantitation and real-time imaging with the TAB monoclonal antibody. Blood 75:390-8, 1990.

13. E. F. Grabowski, and E. Nestoridi. Active site-inhibited rFVIIa (ASIS) blocks platelet adhesion/aggregation on intact endothelium activated with TNFα and Shiga toxin-1. Poster presented at the 2006 Annual Meeting of the American Society of Hematology. Dec. 9, 2006.

14. W. A. Knaus, E. A. Draper, D. P. Wagner, and J. E. Zimmerman. APACHE II: A severity of disease classification system. Critical Care Medicine 13:818-29, 1985.

15. R. I. Kushak, E. Nestoridi, J. W. Lambert, M. K. Selig, J. R. Ingelfinger, and E. F. Grabowski. Detached endothelial cells and microparticles as sources of tissue factor activity. Thrombos. Res. 116:409-19, 2005.

16. Z. Y. Liu, N. H. Loh, S. B. Tor, Y. Murakoshi, R. Maeda, K. A. Khor, and T. Shimidzu. Injection molding of 316L stainless steel microstructures. Microsystems Technologies 9:507-10, 2004.

17. R. P. McEver, N. L. Baenziger, P. W. Majerus. Isolation and quantitation of the platelet membrane glycoprotein deficient in thromboasthenia using a monoclonal hybridoma antibody. J. Clin. Invest. 66:1311-16, 1980.

18. R. P. McEver, E. M. Bennett, and M. N. Martin. Identification of two structurally and functionally distinct sites on human platelet glycoprotein IIB-IIIA using monoclonal antibodies. J Biol. Chem. 258:5269-74, 1983.

19. J. C. Marschall et al. MODS: A reliable descriptor of a complex clinical outcome. Critical Care Medicine 23:1638-1652, 1995.

20. M. M. Pollack et al. PRISM score. Critical Care Medicine 16:1110-1116, 1988.

21. A. Slater, F. Shann, and G. Pearson. PIM2: A revised version of the Paediatric Index of Mortality. Intensive Care Medicine 29:278-285, 2003.

22. T. B. Tschopp, H. J. Weiss, and H. R. Baumgartner. Decreased adhesion of platelets to subendothelium in von Willebrand disease. J Lab Clin Med 83:296-300, 1974.

23. V. Vickerman, J. Blundo, S. Chung, and R. D. Kamm. Design, fabrication and implementation of a novel multi-parameter control microfluidic platform for three-dimensional cell culture and real-time imaging. Lab Chip 8:1468-77, 2008.

24. H. J. Weiss, V. T. Turitto, and H. R. Baumgartner. Effect of shear rate on platelet interaction with subendothelium in citrated and native blood. I. Shear rate-dependent decrease of adhesion in von Willebrand disease and the Bernard-Soulier syndrome. J Lab Clin Med 92:750-64, 1978.

1. A device for assessing coagulation, the device comprising:
a channel configured such that shear stress, in a first portion of the channel, applied to blood drawn through the channel by a vacuum-tube attached to an outlet of the channel approximates physiological shear stress.

2. The device of claim 1, wherein the channel comprises:
a flow chamber; and
a resistance path downstream of the flow chamber, wherein the resistance path is configured such that the shear stress in the flow chamber applied to blood drawn through the channel by the vacuum-tube is between about 1 and 100 dynes per square centimeter.

3. The device of claim 2, wherein the flow chamber has a flow chamber width between about 1 to 8 mm and a flow chamber height between about 200 and 350 microns.

4. The device of claim 2, wherein the resistance path has a resistance path width between about 0.1 and 2 mm and a resistance path height between about 0.1 and 2 mm.

5. The device of claim 4, wherein the resistance path has a length between about 5 and 15 centimeters.

6. The device of claim 1, wherein the channel is configured such that, for healthy subjects, occlusion of the channel by platelet thrombus occurs after less than 0.5 to 5 ml of blood are drawn into the device.

7. The device of claim 1, wherein the channel is configured such that, for subjects in shock, occlusion of the channel by platelet thrombus does not occur before more than 5 ml of blood are drawn into the device.

8. The device of claim 1, wherein the shear stress, in the first portion of the channel, applied to blood drawn through the channel by a vacuum-tube is between about 1 and 100 dynes per square centimeter.

9. The device of claim 1, wherein the shear rate, in a first portion of the channel, applied to blood drawn through the channel by a vacuum-tube is between about 100 and 10,000 per second.

10. The device of claim 1, wherein the channel is configured such that shear stress, in the first portion of the channel, applied to blood drawn through the channel by a vacuum tube whose volume is less than or equal to 10 ml attached to the outlet of the channel approximates physiological shear stress.

11. The device of claim 1, wherein the channel is defined at least in part by a polymer layer bonded to a glass slide.

12. A method comprising:
drawing blood into a channel by attaching a vacuum tube to an outlet of the channel;
observing a length of time that passes until flow of blood through the channel is occluded; and
characterizing a condition of a patient from whom the blood was drawn based at least in part on the length of time that passes until blood flow through the channel is occluded.

13. The method of claim 12, further comprising observing degrees of platelet adhesion and aggregation in the channel until the flow of blood through the channel is occluded.

14. The method of claim 12, further comprising detecting and quantifying tissue factor positive microparticles on platelets attached to surfaces of the channel.

15. The method of claim 12, further comprising comparing the length of time that passes until blood flow through the channel is occluded with a total volume of the vacuum tube.

16. The method of claim 12, wherein drawing blood is limited to drawing less than 5 ml of blood into the device.

17. A kit comprising:
a device of claim 1; and
a vacuum source.

18. The kit of claim 17, wherein the vacuum source is a vacuum tube.

19. The kit of claim 17, further comprising a butterfly needle.

20. The kit of claim 17, further comprising an angiocatheter.
